SEQUENCE LISTING (1 sequence)

SEQ ID NO: 1 - DNA, 2286 bp, Homo sapiens
cccgggaggtaccgagctcttacgcgtgctagctcgagatctcgtggctaaactccaaaagggagggggtataaggaggcgtgtccaacctcccttcctttcatggcctgaaatttagttttttaggtttctttggcccagagggggtccatttagtcagttggggagcttagaacctcatttttagtttacagtaatgagacacagcagatgagtagattgattttattttaagacagagtctcactctgtcacccaggctggagtgagagtggagtggcacaatcacagcccactgcagccccaaccccctggactcaagcaatcctcctgcctcagactcccaagtagctgggacctcaggcacttgccaccatgcctcgctggggtcttttaaattattattatcgtagtgacagggtctccctatgttgcccaggctggtcttgaactcctggcctcaagcgatcctcccacctcagcctccccacgtgctgggtgaaccactgcaaacctggcttcagacgagtggatttaaactttaaatccctcatctgtgccccaactaagtttccatgagtgagcctgtggttactggaatgtgagatttctccaactttctttccttatcattcttcaaattacaacttcagtggagcatttaaaattataattaaaaattccctgagttgatccaaaaaattaaatatctgatattcatgtgcctctatacaaatgggaaattcatctatcctctcccaaataaatatgattggatatcacaagaaccataaaattcctcccttaaggaagattatgagagagaccacacacacacacacagtgcaagctaaagcaaccccgtcttggatgctaatccaccaagttgatgtgttttgttggttttttggttttgttttgagaagagtctcactctgtcgcccaggatagagtgcagtggtgtgatcttgctcactacaacttccacctcctgggttcaagcgattctcctgccttagccacctgagtagctgggattacaggcgtgtgcaccacgcccagctagtttttgtatttttagtagagatggtgttttgttggccaggctgtcatgaacttccggactcaagtgatccacctgcctcagtctcccaaaatgctgggattacaggctgtagctaccacacccggccaaccaagttgacttctgattaaccagcttttagggaaggcctctaagatttccagttatctattgttccttgtgtaaaagtatgtacttaccataaatcctgcccttagcagattcacacagcattcttgcctttccctgggggactgacttcaaatgtccgtcacattcctttcctatagcatataggccctgggcttgggggtaatggcatggggatccaccatcttgtctccctgccgctgaagccagagactatggcttctgttcataaatccctttctccttaaatatgaagtcaaaggtcatgtagataggagctgctgctgaagaaggattttttgtctgaagagttctgtcccctgggcttacttggctatggggtggacccctggccaggagacagcaaactgtttcagtaatgtgcgtgtcgtgcgtgtgtgtgttctgtggtgagaggctatgccattaaacaaacggtgtggctttgggcaagctgctatcttgggacctcagcacactcattcataaattgtgtcttttgggtgagatttgtcttggggtcttcaaaacccctgagactgtggcacatacagagcctaacggcattgaggagggcaagcattcaacagttaggacaatgtgaactgtagctcagctctgctaggtaccttcacaagacctcgaatgctgccccttactatgcctcggttttcttatctataaaacggcattttatcttgttggtggagctgttaaataattaaaagacgtaaagggtctggcaccatggttggcaaaaaatatcagtttcccttctcgacttctcgattttgcccaggaccagtgatgttcacattcaggacctgcctgaaccggcaagccctccacgtgggtccaaagtgcaaagggaaagtcactgctagaggcgccagtaccagcatagcgcccccacgcgccgagtcggggaactgccgcgggggccggccccgcccaccagcgcctgcgcctgcgcagaggcacgccccaagtttgttgtgaccggaagtttgttgtgaccggaagcttggcattccggtactgttggtaaaatg

(19) United States
(12) Patent Application Publication
(10) Pub. No.: US 2006/0228776 A1
(43) Pub. Date: Oct. 12, 2006

(54) PINK-1 promoter

(75) Inventors: Michael Kaplitt, New York, NY (US); Serguei Moussatov, New York, NY (US)

Correspondence Address: NUTTER MCCLENNEN & FISH LLP, WORLD TRADE CENTER WEST, 155 SEAPORT BOULEVARD, BOSTON, MA, 02210-2604, US

(73) Assignee: NEUROLOGIX, INC., Floral Park, NY (US)

(21) Appl. No.: 11/256,372

(22) Filed: Oct. 21, 2005

Related U.S. Application Data
(60) Provisional application No. 60/621,156, filed on Oct. 22, 2004.

(51) Int. Cl. A61K 48/00; C12N 15/85; C07H 21/04; C12P 21/06; C12N 9/12

(52) U.S. Cl. 435/69.1; 435/194; 435/320.1; 435/325; 514/44; 536/23.2

(57) ABSTRACT

The invention provides methods and compositions of an upstream regulatory element (PINK-1 promoter) operably linked to an expressible gene, wherein the expression of the expressible gene is driven by the upstream regulatory element.

### CROSS REFERENCE TO RELATED APPLICATIONS

This application claims priority to U.S. Provisional Patent Application No. 60/621,156 filed on Oct. 22, 2004 and entitled “Pink-1-Promoter.”

### BACKGROUND OF THE INVENTION

The invention is generally in the field of methods and compositions for treating diseases characterized associated with the phosphoinositide 3-kinase (PI 3-kinase)/Akt pathway or the PTEN pathway such as neurological diseases, cardiovascular disorders, endocrine disorders, cancers, and the like. More specifically, the invention pertains to using a regulatory elements such as the PINK-1 promoter, to alter the expression of a gene.

Regulation of gene expression is crucial to the full development of safe and effective gene therapies. While some gene therapy approaches may be effective in the absence of regulation, the ability for genes to turn on and off under specific physiological conditions will facilitate the introduction of many gene therapies which would otherwise be suboptimal in the absence of regulation. One of these areas in particular relates to cell death in disorders such as neurological disease or cardiovascular disease. There are many pathways which have been shown to influence the survival of such cells. Often these can be regulated by a variety of factors, such as growth factors. One such pathway is the PI3Kinase/AKT pathway. Akt is an important cellular survival factor. Increases in Akt have been shown to protect a variety of cells from death, including neurons and myocardial cells. Akt activity can be blocked by a gene called PTEN, although many other factors can potentially regulate Akt activity.

Accordingly, a need exists to develop therapies that can alter the activity of a protein or factor involved in the PI3Kinase/AKT pathway to ameliorate diseases associated with this pathway.

### SUMMARY OF INVENTION

The invention is based on the discovery that diseases associated with the PI-3 kinase/AKT or PHEN pathway can be modified or ameliorated with a chimeric gene construct comprising an upstream regulatory element (PINK-1 promoter) operably linked to an a therapeutic gene, wherein the expression of the therapeutic gene is driven by the upstream regulatory element.

More specifically, one aspect of the invention provides for novel polypeptides. In particular, the invention provides for novel PINK-1 polypeptides. The invention also provides polypeptides that have substantial homology to the foregoing novel polypeptides, modified forms of the novel polypeptides and fragments of the polypeptides. The invention also includes successors or metabolites of the novel polypeptides in biological pathways. The invention also provides molecules that comprise a novel polypeptide, homologous polypeptide, a modified novel polypeptide or a fragment, successor or metabolites. As used herein, the term “polypeptides of the invention” shall be understood to include all of the foregoing.

Another aspect of the invention provides polynucleotides encoding polypeptides of the invention (“novel polynucleotides”). The invention also provides polynucleotides that have substantial homology to novel polynucleotides, modified novel polynucleotides, and fragments of novel polynucleotides. The novel polynucleotides of the present invention are intended to include analogs, compounds having a native polypeptide sequence and structure with one or more amino acid additions, substitutions (generally conservative in nature) and/or deletions, relative to the native molecule, so long as the modifications do not alter the differential expression of the polypeptide. As used herein, the term “polynucleotides of the invention” shall be understood to include all of the foregoing.

Another aspect of the invention provides molecules that specifically bind to a polypeptide of the invention, polynucleotide of the invention or fragments thereof. The binding molecule may be an antibody, antibody fragment, or other molecule. The invention also provides methods for producing a binding molecule that specifically recognizes a polypeptide of the invention, metabolite of the invention or polynucleotide of the invention.

Accordingly, in one aspect, the invention pertains to a promoter sequence comprising SEQ ID NO: 1. Also within the scope of the invention are a promoter sequence comprising fragments, variants and homologous sequences of SEQ ID NO: 1. The promoter sequence may comprise sub-domains of sequences such as those selected from the group consisting of a first NF-kB domain, CRE-BP domain, Interferon Response Stimulated Element, Interferon Regulatory Factor 2, and a second NF-kB domain. The promoter sequence may be an inducible promoter sequence, that may be activated in response to altered Akt levels.

In another aspect, the invention pertains to a promoter-driven protein expression system comprising a PINK-1 promoter sequence operably linked to a therapeutic gene. The therapeutic gene may be the PINK-1 gene.

In yet another aspect, the invention pertains to a plasmid or vector comprising a nucleic acid comprising a PINK-1 promoter sequence operably linked to a therapeutic gene, as well as host cells comprising such plasmids or vectors.

In yet another aspect, the invention pertains to a method of producing a recombinant protein by transforming a host cell with a vector comprising a nucleic acid comprising a PINK-1 promoter sequence operably linked to a therapeutic gene, and expression of the therapeutic gene.

In yet another aspect, the invention pertains to a method for ameliorating a disorder associated with a PI-3 kinase/Akt pathway in a subject by delivering a vector comprising a PINK-1 promoter operably linked to a therapeutic gene to the target site in the subject, an expressing the therapeutic gene in the target site to ameliorate the disorder. Examples of disorder associated with a PI-3 kinase/Akt pathway include, but are not limited to, cardiovascular disorders, neurodegenerative disorders, cell proliferative disorders, cancers, and endocrine disorders.

### BRIEF DESCRIPTION OF THE FIGURES

FIG. 1A illustrates PINK1 levels in stable PC12 cell lines overexpressing PTEN.

FIG. 1B illustrates PINK1 levels in stable U87 cell lines overexpressing PTEN.

FIG. 2 shows the sequence of the HUMAN PINK1 PROMOTER (PINK1pr2220).

FIG. 3A illustrates the effect of PTEN on isolated PINK1 promoter fragment. Level of firefly luciferase activity driven by the PINK1 promoter normalized against renilla luciferase activity from a second plasmid used to control for transfection variability.

FIG. 3B illustrates the effect of PTEN on isolated PINK1 promoter fragment. Level of activated, phosphorylated AKT on Western blot.

FIG. 3C illustrates the effect of PTEN on isolated PINK1 promoter fragment. Measurement of PINK1 promoter activity.

FIG. 4 shows that the co-transfection of a dominant-negative AKT (dnAKT) mutant induced PINK1 promoter activity to levels comparable to PTEN. Although PTEN blockade did not inhibit PINK1 promoter activity, co-transfection with a consiitutively-active AKT (cAKT) did reduce PINK1 promoter activity roughly 2-fold.

FIG. 5 illustrates that co-transfection of both cAKT and PTEN resulted in PINK1 promoter activity which was below baseline near levels resulting from cAKT transfection alone.

FIG. 6A illustrates 6-OHDA reduced cell viability of human neuroblastoma SH-SY5Y in a dose dependent manner.

FIG. 6B is a Western blot analysis of cells treated at various time points with PTEN and phosphorylated PTEN (p-PTEN).

FIG. 6C shows a lower p-PTEN/total PTEN ratio for cells treated with 50 mM of 6-OHD for 6 hours, indicative of higher PTEN activity.

FIG. 6D illustrates p-PTEN/total PTEN ratio of the subtantia nigra of 6-OHDA unilaterally lesioned rats.

FIG. 7A is a Western Blot analysis showing a PTEN siRNA adenoviral associated viral vector construct reducing PTEN levels in SH-SKN cells.

FIG. 7B is a quantitative PCR measurement of the reduction of PTEN mRNA levels by the same construct used to produce the Western Blot of FIG. 7A.

FIG. 7C illustrates a reduction in apoptosis in SH-SY5Y cells transfected with two separate PTEN siRNA plasmids compared to scrambled siRNA control.

FIG. 8A shows a Western which illustrates a reduction in PTEN protein levels in SH-SY5Y cells transfected with SMARTpool siRNA reagent.

FIG. 8B shows reduced cleavage of the Caspace-3 active metabolite in SH-SY5Y cells which had been transfected with PTEN RNAi and challenged with 6-OHDA for 6 hours.

FIG. 8C shows increased cell viability of SH-SY5Y cells transfected with PTEN siRNA oligos.

FIG. 9A shows dose dependent reduction of cell viability of human neuroblastoma SH-SY5Y when treated with MPP+ for 24 hours.

FIG. 9B is a Western Blot of SH-SY5Y cells treated for 24 hours with escalating (high) doses of MPP+ showing the ratio of p-PTEN/total PTEN.

FIG. 9C is a Western Blot of SH-SY5Y cells treated for with escalating low doses of MPP+ showing the ratio of p-PTEN/total PTEN.

### DETAILED DESCRIPTION

The practice of the present invention employs, unless otherwise indicated, conventional methods of virology, microbiology, molecular biology and recombinant DNA techniques within the skill of the art. Such techniques are explained fully in the literature. (See, e.g., Sambrook, et al. Molecular Cloning: A Laboratory Manual (Current Edition); DNA Cloning: A Practical Approach, Vol. I & II (D. Glover, ed.); Oligonucleotide Synthesis (N. Gait, ed., Current Edition); Nucleic Acid Hybridization (B. Hames & S. Higgins, eds., Current Edition); Transcription and Translation (B. Hames & S. Higgins, eds., Current Edition); CRC Handbook of Parvoviruses, Vol. I & II (P. Tijessen, ed.); Fundamental Virology, 2nd Edition, Vol. I & II (B. N. Fields and D. M Knipe, eds.)).

So that the invention is more clearly understood, the following terms are defined:

The phrase “a disorder associated with the PI-3 kinase/akt pathway” or “a disease associated with PI-3 kinase/akt pathway” as used herein refers to any disease state associated with the PI-3 kinase/akt pathway or any cell receptor involved with the pathway, e.g., the akt receptor. These phrases are also intended to include disorders or diseases in which akt influences the cellular physiology and/or etiology of the disease. Examples of such disease include, but are not limited to, cardiovascular disorders (e.g. a cardiac disease that functions through the akt receptor), neurodegenerative disorders, cell proliferative disorders, diseases associated with angiogenesis, cancers, endocrine disorders (e.g., endocrine disorders that involve activation of akt such as diabetes), and the like.

The phrase “a disorder associated with the PTEN pathway” or “a disease associated with PTEN pathway” as used herein refers to any disease state associated with the PTEN pathway or any cell receptor involved with the pathway. These phrases are also intended to include disorders or diseases in which PTEN influences the cellular physiology and/or etiology of the disease. Examples of such disease include, but are not limited to, cardiovascular disorders (e.g. a cardiac disease), neurodegenerative disorders, cell proliferative disorders, diseases associated with angiogenesis, cancers, endocrine disorders (e.g., diabetes), and the like.

The term “homology” or “identity” as used herein refer to the percent identity of two amino acid sequences or of two nucleic acid sequences. To determine the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, or 90% of the length of the reference sequence (e.g., when aligning a second sequence to the sequence of SEQ ID NO: 1. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

As used herein, two polypeptides are “substantially homologous” when there is at least 70% homology, at least 80% homology, at least 90% homology, at least 95% homology or at least 99% homology between their amino acid sequences, or when polynucleotides encoding the polypeptides are capable of forming a stable duplex with each other. Likewise, two polynucleotides are “substantially homologous” when there is at least 70% homology, at least 80% homology, at least 90% homology, at least 95% homology or at least 99% homology between their amino acid sequences or when the polynucleotides are capable of forming a stable duplex with each other. In general, “homology” refers to an exact nucleotide-to-nucleotide or amino acid-to-amino acid correspondence of two polynucleotides or polypeptide sequences, respectively.

The comparison of sequences and determination of percent identity between two sequences can be accomplished using a mathematical algorithm. In a preferred embodiment, the percent identity between two amino acid sequences is determined using the Needleman and Wunsch (J. Mol. Biol. (48):444-453 (1970)) algorithm which has been incorporated into the GAP program in the GCG software package (available at http://www.gcg.com), using either a Blosum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at http:/lwww.gcg.com), using a NWSgapdna.CMP matrix and a gap weight of 40, 50, 60, 70, or 80 and a length weight of 1, 2, 3, 4, 5, or 6. In another embodiment, the percent identity between two amino acid or nucleotide sequences is determined using the algorithm of E. Meyers and W. Miller (CABIOS, 4:11-17 (1989)) which has been incorporated into the ALIGN program (version 2.0), using a PAM120 weight residue table, a gap length penalty of 12 and a gap penalty of 4.

The nucleic acid and protein sequences of the present invention can further be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to PINK-1 nucleic acid molecules of the invention. BLAST protein searches can be performed with the XBLAST program, score=50, wordlength=3 to obtain amino acid sequences homologous to PINK-1 protein molecules of the invention. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used. See http:H/www.ncbi.nlm.nih.gov.

The terms “neurological disorder” or “neurodegenerative disorder” are used interchangeably herein and refer to an impairment or absence of a normal neurological function or presence of an abnormal neurological function in a subject. For example, neurological disorders can be the result of disease, injury, and/or aging. As used herein, neurological disorder also includes neurodegeneration which causes morphological and/or functional abnormality of a neural cell or a population of neural cells. Non-limiting examples of morphological and functional abnormalities include physical deterioration and/or death of neural cells, abnormal growth patterns of neural cells, abnormalities in the physical connection between neural cells, under- or over production of a substance or substances, e.g., a neurotransmitter, by neural cells, failure of neural cells to produce a substance or substances which it normally produces, production of substances, e.g., neurotransmitters, and/or transmission of electrical impulses in abnormal patterns or at abnormal times. Neurodegeneration can occur in any area of the brain of a subject and is seen with many disorders including, for example, Amyotrophic Lateral Sclerosis (ALS), multiple sclerosis, Huntington's disease, Parkinson's disease, and Alzheimer's disease.

The term “operably linked” as used herein refers to an arrangement of elements wherein the components are configured so as to perform their usual function. Thus, control elements operably linked to a coding sequence are capable of effecting the expression of the coding sequence, so long as they function to direct the expression of the coding sequence. For example, intervening untranslated yet transcribed can be present between a promoter sequence and the coding sequence and the promoter sequence can still be considered “operably linked” to the coding sequence.

“Nucleic acid” or a “nucleic acid molecule” as used herein refers to any DNA or RNA molecule, either single or double stranded and, if single stranded, the molecule of its complementary sequence in either linear or circular form. In discussing nucleic acid molecules, a sequence or structure of a particular nucleic acid molecule may be described herein according to the normal convention of providing the sequence in the 5′ to 3′ direction. With reference to nucleic acids of the invention, the term “isolated nucleic acid” is sometimes used. This term, when applied to DNA, refers to a DNA molecule that is separated from sequences with which it is immediately contiguous in the naturally occurring genome of the organism in which it originated. For example, an “isolated nucleic acid” may comprise a DNA molecule inserted into a vector, such as a plasmid or virus vector, or integrated into the genomic DNA of a prokaryotic or eukaryotic cell or host organism.

When applied to RNA, the term “isolated nucleic acid” refers primarily to an RNA molecule encoded by an isolated DNA molecule as defined above. Alternatively, the term may refer to an RNA molecule that has been sufficiently separated from other nucleic acids with which it would be associated in its natural state (i.e., in cells or tissues). An isolated nucleic acid (either DNA or RNA) may further represent a molecule produced directly by biological or synthetic means and separated from other components present during its production. “Natural allelic variants”, “mutants” and “derivatives” of particular sequences of nucleic acids refer to nucleic acid sequences that are closely related to a particular sequence but which may possess, either naturally or by design, changes in sequence or structure. By closely related, it is meant that at least about 75%, but often, more than 90%, of the nucleotides of the sequence match over the defined length of the nucleic acid sequence referred to using a specific SEQ ID NO. Changes or differences in nucleotide sequence between closely related nucleic acid sequences may represent nucleotide changes in the sequence that arise during the course of normal replication or duplication in nature of the particular nucleic acid sequence. Other changes may be specifically designed and introduced into the sequence for specific purposes, such as to change an amino acid codon or sequence in a regulatory region of the nucleic acid. Such specific changes may be made in vitro using a variety of mutagenesis techniques or produced in a host organism placed under particular selection conditions that induce or select for the changes. Such sequence variants generated specifically may be referred to as “mutants” or “derivatives” of the original sequence.

The present invention also includes methods of use for active portions, fragments, derivatives and functional or non-functional mimetics of the PINK-1 promoter.

A “fragment” or “portion” means a continguous stretch of nucleotides or amino acid residues of at least about five to seven contiguous amino acids, often at least about seven to nine contiguous amino acids, typically at least about nine to thirteen contiguous amino acids and, most preferably, at least about twenty to thirty or more contiguous nucleotides or amino acids.

The term “subject” as used herein refers to any living organism capable of eliciting an immune response. The term subject includes, but is not limited to, humans, nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be covered.

The invention is described in more detail in the following subsections:

I. Diseases Associated with the PI-3 Kinase/Akt Pathway or the PTEN Pathway

In one aspect, the invention pertains to ameliorating diseases or disorders associated with the PI-3 kinase/akt pathway, in which akt influences the cellular physiology and/or etiology of the disease. The invention also pertains to ameliorating diseases or disorders associated with the PTEN pathway. Examples of such disease include, but are not limited to, cardiovascular disorders (e.g. a cardiac disease that functions through the akt receptor), neurodegenerative disorders, cell proliferative disorders, diseases associated with angiogenesis, cancers, endocrine disorders (e.g., endocrine disorders that involve activation of akt such as diabetes), and the like.

Examples of Neurodegenerative diseases are as follows:

Huntington's disease (HD) is a hereditary disorder caused by the degeneration of neurons in certain areas of the brain. This degeneration is genetically programmed to occur in certain areas of the brain, including the cells of the basal ganglia, the structures that are responsible for coordinating movement. Within the basal ganglia, Huntington's disease specifically targets nerve cells in the striatum, as well as cells of the cortex, or outer surface of the brain, which control thought, perception and memory. Neuron degeneration due to HD can result in uncontrolled movements, loss of intellectual capacity and faculties, and emotional disturbance, such as, for example, mood swings or uncharacteristic irritability or depression.

As discussed above, neuron degeneration due to HD is genetically programmed to occur in certain areas of the brain. Studies have shown that Huntington's disease is caused by a genetic defect on chromosome 4, and in particular, people with HD have an abnormal repetition of the genetic sequence CAG in the HD gene, which has been termed IT15. The IT15 gene is located on the short arm of chromosome 4 and encodes a protein called huntingtin. Exon I of the IT15 gene contains a polymorphic stretch of consecutive glutamine residues, known as the polyglutamine tract (D. Rubinsztein, “Lessons from Animal Models of Huntington's Disease,” TRENDS in Genetics, 18(4): 202-9 (April 2002)). Asymptomatic individuals typically contain fewer than 35 CAG repeats in the polyglutamine tract.

The inherited mutation in HD is an expansion of the natural CAG repeats within the sequence of exon 1 of the human HD gene. This leads to an abnormally long stretch of polyglutamines. The length of the polyglutamine repeats correlates with the severity of the disease. One of the pathological hallmarks of HD is a buildup of intracellular protein aggregates composed of these abnormal HD proteins with long polyglutamine repeats. The results in the Examples section show that expression of this abnormal HD gene (called Huntington) in cultured neurons leads to cell death, while co-expression of the anti-apoptotic gene XIAP blocks this death. This demonstrates that expression of an anti-apoptotic gene can protect from mutant Huntington-induced neuronal death.

(b) Multiple Sclerosis

Multiple Sclerosis (MS) is a chronic disease that is characterized by “attacks,” during which areas of white matter of the central nervous system, known as plaques, become inflamed. Inflammation of these areas of plaque is followed by destruction of myelin, the fatty substance that forms a sheath or covering that insulates nerve cell fibers in the brain and spinal cord. Myelin facilitates the smooth, high-speed transmission of electrochemical messages between the brain, spinal cord, and the rest of the body. Damage to the myelin sheath can slow or completely block the transmission of these electrochemical messages, which can result in diminished or lost bodily function.

The most common course of MS manifests itself as a series of attacks, which are followed by either complete or partial remission, during which the symptoms lessen only to return at some later point in time. This type of MS is commonly referred to as “relapsing-remitting MS.” Another form of MS, called “primary-progressive MS,” is characterized by a gradual decline into the disease state, with no distinct remissions and only temporary plateaus or minor relief from the symptoms. A third form of MS, known as “secondary-progressive MS,” starts as a relapsing-remitting course, but later deteriorates into a primary-progressive course of MS.

The symptoms of MS can be mild or severe, acute or of a long duration, and may appear in various combinations. These symptoms can include vision problems such as blurred or double vision, red-green color distortion, or even blindness in one eye, muscle weakness in the extremities, coordination and balance problems, muscle spasticity, muscle fatigue, paresthesias, fleeting abnormal sensory feelings such as numbness, prickling, or “pins and needles” sensations, and in the worst cases, partial or complete paralysis. About half of the people suffering from MS also experience cognitive impairments, such as for example, poor concentration, attention, memory and/or judgment. These cognitive symptoms occur when lesions develop in those areas of the brain that are responsible for information processing.

(c) Alzheimer's Disease

Alzheimer's disease is a progressive, neurodegenerative disease that affects the portions of the brain that control thought, memory and language. This disease is characterized by progressive dementia that eventually results in substantial impairment of both cognition and behavior. The disease manifests itself by the presence of abnormal extracellular protein deposits in brain tissue, known as “amyloid plaques,” and tangled bundles of fibers accumulated within the neurons, known as “neurofibrillary tangles,” and by the loss of neuronal cells. The areas of the brain affected by Alzheimer's disease can vary, but the areas most commonly affected include the association cortical and limbic regions. Symptoms of Alzheimer's disease include memory loss, deterioration of language skills, impaired visuospatial skills, and impaired judgment, yet those suffering from Alzheimer's retain motor function.

(d) Parkinson's Disease

Parkinson's disease (PD) is characterized by death of dopaminergic neurons in the substantia nigra (SNr), leading to a disturbance in the basal ganglia network which regulates movement. In addition, other brainstem cell populations can die or become dysfunctional. One of the pathological hallmarks of PD in humans is the Lewy body, which contains abnormal protein aggregates which include the protein alpha-synuclein. While there are many therapies available to treat the symptoms of Parkinson's disease, including medical therapy and surgical therapies, there is no current treatment which will stop the death of neurons and ultimately cure this disorder.

To date, the cause of neuronal death has remained elusive. One problem has been the relevance of current animal models to human disease. The gold-standard animal models for PD involve rapid destruction of dopamine neurons using chemicals which are fairly specific for dopamine neurons. These chemical toxins, which include 6-hydroxydopamine (6OHDA) and MPTP, cause oxidative damage to dopamine neurons in both rodents and primates. These models can be useful to test the efficacy of new therapies designed to improve the symptoms of PD, since such treatments are designed to intervene after cells have died or become dysfunctional, regardless of the cause of cell death. In order to test the value of protective or curative strategies, however, the mechanism of cell death must be relevant to human disease otherwise successful experimental studies will not translate into effective human therapy.

Many features of the animal models have been questioned for protective strategies. First, these toxins usually cause near complete destruction of dopamine neurons within 24-48hrs., while PD is a slowly degenerative disease which can take many years or more to have even partial loss of cells. Also, these do not cause protein inclusions similar to the Lewy bodies seen in human PD. These toxins are also only specific to dopamine neurons, while in human PD other cell populations are affected. There is also little convincing evidence in human disease that the oxidative damage mechanism is the primary cause of PD. Nonetheless, several factors have been shown to protect animal cells from these toxins, including anti-apoptotic genes and growth factors such as glial-derived neurotrophic factor (GDNF). This is understandable, since the result of such oxidative damage is usually apoptotic cell death.

The history of GDNF highlights the problems in translating promising data from these models to human disease. Several animal studies over many years suggested that GDNF could afford substantial protection to dopamine neurons when exposed to either 6OHDA or MPTP. Similar data has been obtained regardless of the mode of delivery of GDNF, including both intraventricular and direct intrastriatal infusion of recombinant GDNF protein, as well as GDNF produced from a viral vector following gene therapy. Nonetheless, multiple GDNF studies in human have failed. The first studies involved infusion of GDNF into cerebrospinal fluid via an intraventricular catheter. This was stopped due to adverse effects. It was then hypothesized that direct infusion of GDNF into the striatum, where dopamine neuron terminals reside, would limit side effects and improve efficacy as was seen in the above mentioned animal models. This was also recently halted due to failure to demonstrate any meaningful effect in human patients compared to controls. This only serves to highlight problems with developing neuroprotective therapeutics using these models. In fact, the only similarity between these models and human PD is the loss of dopamine neurons. This, however, can also be achieved by many other means, including thermal destruction or destruction of these cells using other chemicals such as ibotinic acid. Therefore, there is no good evidence that any protection of neurons using these models has any value to human PD.

Recently, a new model was described which not only appears to be more relevant to human PD, but which also is consistent with most of the known features of human disease (Kevin et al, Annals of Neurology (2004) 56, 149-162). The model involves repeated administration of a proteasome inhibitor. Proteasomes are complex, multi-unit enzymes within the cell which are critical for metabolizing and removing proteins which are misfolded, dysfunctional and/or no longer desirable. These are essential for protein turnover, which is crucial for proper regulation of cellular physiology. Proteins which are targeted to the proteasome are usually modified by addition of a ubiquitin group. Ubiquinated proteins can then enter the proteasome for ultimate degradation. Unlike the dopamine toxin model, this model causes a very slow neuronal degeneration which is much more analogous to human disease. In addition to dopamine neuronal loss in the SNr, loss or dysfunctional of other neuronal populations are seen which also mimic the human disorder. Most interestingly, intracellular protein aggregates are seen which are highly analogous to the Lewy body. None of these features are present in the dopamine toxin models, and all of them are found to some degree in the human disorder, indicating that this is a far more relevant model of the actual mechanism of cell death in human PD.

Those few forms of human PD for which a cause is known further support the relevance of this model for neuroprotection studies. A minority of PD cases are caused by inherited mutations in a single gene. To date, four such genes have been identified. While the function of one gene remains unknown, the other three directly support the concept that ubiquitin-proteasome dysfunction is the key cause of cell death PD. Two of these genes, parkin and UCHL-1, are involved in ubiquination of proteins and loss of function causes human PD. The third gene, alpha-synuclein, causes a dominant form of PD and, as mentioned earlier, is a key component of the intracellular inclusions called Lewy bodies. Therefore, the major known causes of inherited human PD support the pathological findings in the new proteasome inhibitor model of PD as being the only available model which accurately replicates the human disorder.

(e) Amyotrophic Lateral Sclerosis

Amyotrophic Lateral Sclerosis (ALS) is a universally fatal neurodegenerative condition in which patients progressively lose all motor function—unable to walk, speak, or breathe on their own, ALS patients die within two to five years of diagnosis. The incidence of ALS increases substantially in the fifth decade of life. Evidence is accumulating that as a result of the normal aging process the body increasingly loses the ability to adequately degrade mutated or misfolded proteins.

The cardinal feature of ALS is the loss of spinal motor neurons, which causes the muscles under their control to weaken and waste away leading to paralysis. ALS has both familial (5-10%) and sporadic forms and the familial forms have now been linked to several distinct genetic loci (Deng, H. X., et al., “Two novel SOD1 mutations in patients with familial amyotrophic lateral sclerosis,” Hum. Mol. Genet., 4(6): 1113-16 (1995); Siddique, T. and A. Hentati, “Familial amyotrophic lateral sclerosis,” Clin. Neurosci., 3(6): 338-47(1995); Siddique, T., et al., “Familial amyotrophic lateral sclerosis,” J. Neural Transm. Suppl., 49: 219-33(1997); Ben Hamida, et al., “Hereditary motor system diseases (chronic juvenile amyotrophic lateral sclerosis). Conditions combining a bilateral pyramidal syndrome with limb and bulbar amyotrophy,” Brain, 113(2): 347-63 (1990); Yang, Y., et al., “The gene encoding alsin, a protein with three guanine-nucleotide exchange factor domains, is mutated in a form of recessive amyotrophic lateral sclerosis,” Nat. Genet., 29(2): 160-65 (2001); Hadano, S., et al., “A gene encoding a putative GTPase regulator is mutated in familial amyotrophic lateral sclerosis 2,” Nat. Genet., 29(2): 166-73 (2001)). About 15-20% of familial cases are due to mutations in the gene encoding Cu/Zn superoxide dismutase 1 (SODi) (Siddique, T., et al., “Linkage of a gene causing familial amyotrophic lateral sclerosis to chromosome 21 and evidence of genetic-locus heterogeneity,” N. Engl. J. Med., 324(20): 1381-84 (1991); Rosen, D. R., et al., “Mutations in Cu/Zn superoxide dismutase gene are associated with familial amyotrophic lateral sclerosis.” Nature, 362(6415): 59-62 (1993)).

Although the etiology of the disease is unknown, the dominant theory is that neuronal cell death in ALS is the result of over-excitement of neuronal cells due to excess extracellular glutamate. Glutamate is a neurotransmitter that is released by glutaminergic neurons, and is taken up into glial cells where it is converted into glutamine by the enzyme glutamine synthetase, glutamine then re-enters the neurons and is hydrolyzed by glutaminase to form glutamate, thus replenishing the neurotransmitter pool. In a normal spinal cord and brain stem, the level of extracellular glutamate is kept at low micromolar levels in the extracellular fluid because glial cells, which function in part to support neurons, use the excitatory amino acid transporter type 2 (EAAT2) protein to absorb glutamate immediately. A deficiency in the normal EAAT2 protein in patients with ALS, was identified as being important in the pathology of the disease (See e.g., Meyer et al., J. Neurol. Neurosurg. Psychiatry, 65: 594-596 (1998); Aoki et al., Ann. Neurol. 43: 645-653 (1998); Bristol et al., Ann Neurol. 39: 676-679 (1996)). One explanation for the reduced levels of EAAT2 is that EAAT2 is spliced aberrantly (Lin et al., Neuron, 20: 589-602 (1998)). The aberrant splicing produces a splice variant with a deletion of 45 to 107 amino acids located in the C-terminal region of the EAAT2 protein (Meyer et al., Neureosci Lett. 241: 68-70 (1998)). Due to the lack of, or defectiveness of EAAT2, extracellular glutamate accumulates, causing neurons to fire continuously. The accumulation of glutamate has a toxic effect on neuronal cells because continual firing of the neurons leads to early cell death.

Other example of diseases associated with the PI-3 kinase/akt or PTEN pathway are cardiovascular disorders such as heart failure, ischemic heart disease, and cardiotoxicity. Examples of endocrine diseases are those that result form an impairment or absence of a normal endocrine function or presence of an abnormal endocrine function in a subject. For example, endocrine disorders can be characterized by the disturbance in the regulation of mood, behavior, control of feeding behavior and production of substances, such as insulin in diabetes. Also included are disease involving growth factors that are influenced by either the PI-3 kinase/akt or PTEN pathway e.g, VEGF in angiogenesis.

II. The PTEN and PI-3 Kinase/AKT Pathways

(i) The PTEN Pathway

PTEN is a tumor suppressor gene that is able to dephosphorylate phosphatidylinositol 3,4,5-trisphosphate (PI-3,4,5-P3), the product of phosphatidyl inositol 3-kinase (PIK). Many of the mutations that have arisen in cancer cells have been mapped to the phosphatase catalytic domain of PTEN. Data suggests that the phosphatase activity of PTEN is essential for its function as a tumor suppressor. The activation of Akt/PKB is regulated by the phosphorylation of Akt on Thr308 and Ser473 by phosphoinositide-dependent kinase (PDK) and integrin-linked kinase (ILK), respectively. Inactivation of PTEN allows constitutive and unregulated activation of the Akt/PKB signaling pathway. In addition to regulating the Akt/PKB signaling pathway, PTEN also inhibits growth factor (GF)-induced Shc phosphorylation and suppresses the MAP kinase signaling pathway. PTEN interacts directly with FAK and is able to dephosphorylate activated FAK. PTEN-induced down-regulation of p130CAS through FAK results in inhibition of cell migration and spreading.

The product of the tumor suppressor gene PTEN was identified as a dual specificity phosphatase and has been shown to dephosphorylate inositol phospholipids in vivo (Li et al Science 1997, Steck et al 1997, Li et al Cancer Res 1997,Myers et al, 1997, Myers et al 1998, Maehama et al, 1998, Stambolic et al 1998, Wu et al 1998). The PTEN gene, which is located on the short arm of chromosome 10 (10q23), is mutated in 40-50% of high grade gliomas as well as many other tumor types, including those of the prostate, endometrium, breast, and lung (Li et al, Science 1997, Steck et al 1997, Maier et al 1998). In addition, PTEN is mutated in several rare autosomal dominant cancer predisposition syndromes, including Cowden disease, Lhermitte-Duclos disease and Bannayan-Zonana syndrome (Liaw et al 1997, Myers et al AJHG 1997, Maehama et al TCB 1999, Cantley and Neel 1999). Furthermore, the phenotype of PTEN-knockout mice revealed a requirement for this phosphatase in normal development and confirmed its role as a tumor suppressor (Podsypanina et al PNAS 1999, Suzuki et al Curr Biol 1998, Di Christofano et al Nat Gen 1998).

PTEN is a 55 kDa protein comprising an N-terminal catalytic domain, identified as a segment with homology to the cytoskeletal protein tensin and containing the sequence HC(X).sub.5 R (SEQ ID NO: 22), which is the signature motif of members of the protein tyrosine phosphatase family, and a C-terminal C2 domain with lipid-binding and membrane-targeting functions (Lee et al Cell 1999). The sequence at the extreme C-terminus of PTEN is similar to sequences known to have binding affinity for PDZ domain-containing proteins. PTEN is a dual specificity phosphatase that displays a pronounced preference for acidic substrates (Myers et al PNAS 1997). Importantly, PTEN possesses lipid phosphatase activity, preferentially dephosphorylating phosphoinositides at the D3 position of the inositol ring. It is one of two enzymes known to dephosphorylate the D3 position in inositol phospholipids. PTEN phosphatase activity has also been implicated in many cellular biochemical reactions. It is an object of the invention to also provide methods for the identification of agents which impact PTEN modulation of immunoreceptors, AKT, P13 kinase and p53 signaling. PTEN is an important signaling molecule which modulates a wide variety of cellular processes. These cellular processes include angiogenesis, cellular migration, immunoreceptor modulation, p53 signaling and apoptotic cell death, P13 and AKT signaling.

The data in the Examples section shows that PTEN mediates at least in part the effects of the neurotoxin 6-hydroxydopamine (6-OHDA), which specifically causes the death of dopaminergic neurons in vivo and in vitro. Rats lesioned with 6-OHDA in the medial forebrain bundle have decreased levels of phosphorylated PTEN (P-PTEN) in the SNc when compared with saline controls. Furthermore, human neuroblastoma SH-SY5Y cells challenged with 6-OHDA showed a similar reduction in P-PTEN by both western blot and immunoprecipitation.

Since phosphorylation inhibits PTEN activity, this suggests that the 6-OHDA insult increased PTEN activity. These changes correlated directly with both the increase in caspase activation at 6 hrs and eventual cell death at 24 hrs. Inhibition of endogenous PTEN using RNA interference (RNAi) resulted in increased cell survival and decreased apoptosis at every dose of 6-OHDA compared with matched controls. For in vivo manipulation, we have now generated an adeno-associated Virus vector containing the PTEN RNAi construct, which appears to reduce PTEN mRNA levels by almost 90%.

These data suggest that alterations in activity of the PTEN tumor suppressor may mediate some of the neurotoxic effects of 6-OHDA, and strategies to block PTEN expression or function in dopaminergic neurons may provide novel gene therapy for Parkinson s disease.

(ii) The Akt Pathway

The serine/threonine protein kinase Akt/PKB is the cellular homologue of the viral oncogene v-Akt and is activated by various growth and survival factors. In mammals, there are three known isoforms of the Akt kinase, Akt1, Akt2, and Akt3. Many cell surface receptors induce the production of second messengers that activate phosphoinositide 3-kinase (PI3K). Akt is located downstream of PI3K and, therefore, functions as part of a wortmannin-sensitive signaling, pathway. PI3K generates phosphorylated phosphatidylinositides (PI-3,4-P2 and PI-3,4,5-P3) in the cell membrane that bind to the amino-terminal pleckstrin homology (PH) domain of Akt. PI-3,4-P2 and PI-3,4,5-P3 also activate phosphoinositide-dependent kinase (PDK) which phosphorylates Thr308 of membrane-bound Akt. Ser473 is phosphorylated by integrin-linked kinase (ILK). Activated Akt promotes cell survival through two distinct pathways: 1) Akt inhibits apoptosis by phosphorylating the Bad component of the Bad/Bcl-XL complex. Phosphorylated Bad binds to 14-3-3 causing dissociation of the Bad/Bcl-XL complex and allowing cell survival. 2) Akt activates IKK-a that ultimately leads to NF-kb activation and cell survival.

III. PINK-1

PINK1 is newly identified cause of the PARK6 form of adult early onset Parkinson's disease. Although this is an autosomal recessive disorder, it has been suggested that heterozygosity leading to haploinsufficiency may represent a risk factor for sporadic Parkinson's disease. Prior to this discovery, little was known about PINK1. The acronym stands for Pten INduced putative Kinase, based upon the only two features about this factor which are known to date. Although the function of the PINK1 gene is unknown, it is believed to be a possible kinase based upon a consensus domain present within the protein sequence. A PINK1 fusion protein also appears to at least partially localize to the mitochondria in cultured cells, suggesting that it may influence mitochondrial function. PINK1 was originally identified as a novel gene induced by the PTEN anti-oncogene in ovarian cancer cells. PTEN is among the most frequently mutated genes in a variety of cancers. PTEN appears to primarily function as a lipid phosphatase, opposing the PI3Kinase pathway and inhibiting activity of the cell survival factor AKT. Nonetheless, other functions have been associated with PTEN, including a protein phosphatase function and direct interaction with and activation of transcription factors such as p53. Recently, we reported that PTEN can block differentiation of cultured neuron-like cells by altering expression of several neuronal genes. Some of these changes were mediated by inhibition of PI3K/AKT, while other changes appeared to be independent of this pathway. In order to better understand the regulation of PINK1 expression, the current study was designed to determine if and how PTEN regulates PINK1 expression, and to identify a possible human PINK1 promoter which would regulate such changes.

Regulation of gene expression is crucial to the full development of safe and effective gene therapies. While some gene therapy approaches may be effective in the absence of regulation, the ability for genes to turn on and off under specific physiological conditions will facilitate the introduction of many gene therapies which would otherwise be suboptimal in the absence of regulation. One of these areas in particular relates to cell death in disorders such as neurological disease or cardiovascular disease. There are many pathways which have been shown to influence the survival of such cells. Often these can be regulated by a variety of factors, such as growth factors. One such pathway is the PI3Kinase/AKT pathway. Akt is an important cellular survival factor. Increases in Akt have been shown to protect a variety of cells from death, including neurons and myocardial cells. Akt activity can be blocked by a gene called PTEN, although many other factors can potentially regulate Akt activity.

Recently, a new gene was identified which causes a rare, inherited form of Parkinson's disease (PD) (Valente et al. (2004) Science, 304: 1158-1160). This gene, called PINK1, was originally cloned from human ovarian cancer cells overexpressing PTEN. Although the function of PINK1 is unknown, the gene appeared to be induced by PTEN. Nonetheless, the mechanism of induction was unclear. First, it was possible that mRNA or protein levels could be increased either by regulation of the unknown PINK1 promoter, or alternatively by regulation of PINK1 mRNA stability. Second, the nature of PTEN regulation was unclear. Although the major function of PTEN appears to be inhibition of Akt activity, other functions have been identified as well. For example, PTEN has been shown to bind to and directly activate the p53 transcription factor. Also, PTEN can act as a protein kinase to phosphorylate certain proteins.

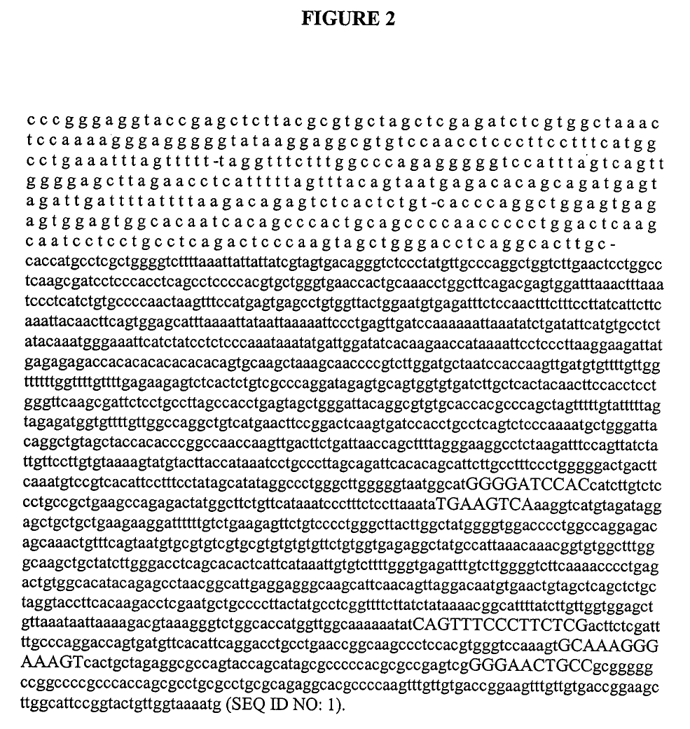

In order to better understand this relationship, the human promoter for PINK1 was cloned. Two different possible start sites for the PINK1 mRNA have been suggested, and based upon this we analyzed the human genome sequence upstream of the most 5-prime known PINK1 mRNA sequence. We then generate oligonucleotide primers and used PCR to clone a 2200 bp fragment of human DNA immediately upstream of the PINK1 mRNA (See FIG. 2, SEQ ID NO: 1). The results from the experiments evaluating the effect of PINK-1 are shown in the Examples section.

This data demonstrates that the PINK-1 sequence can be used in a gene therapy vector to regulate expression of other genes in response to alterations in Akt levels. This provides a mechanism for autoregulation of gene expression in at-risk cells following gene therapy. When Akt activity is low, gene expression would be high, while high levels of Akt activity would block gene expression.

IV. Regulation

Control of gene expression underlies, at some level, all cellular and/or organismal processes, including direction of the development of the organism and cellular responses to outside signals. Gene control occurs at several points in the cellular response, including the activation or suppression of transcription, the differential processing and stabilization of messenger RNA (mRNA), and the extent of translation of the mRNA. Control of transcription plays a particularly critical role in the regulation of gene expression in eukaryotic cells. (See generally, Darnell et al., 1990, Molecular Cell Biology, 2d ed., Chapter 11, W.H. Freeman & Co., NY, pp. 391-448).

Cellular mechanisms mediate the activation of transcription of specific genes, for example, the activation of transcription elicited during development and that elicited by extracellular signals such as hormones or growth factors. In particular, transcription of a specific mRNA coding for a particular gene product is controlled by a set of transcription factor proteins. These proteins bind specific DNA sequences, either promoter or enhancer elements, and form multimeric complexes which activate transcription (Tjian and Maniatis, 1994, Cell 77:5-8). The multitude and cell specificity of the transcription factors and corresponding DNA binding sites allow for the precise regulation of transcription. Thus, the regulation of transcription activation would provide a precise and specific method for controlling the production of particular proteins.

In one aspect, the invention pertains to altering the expression of a protein by a regulatable promoter. Functional analysis of cellular proteins is greatly facilitated through changes in the expression level of the corresponding gene for subsequent analysis of the accompanying phenotype. For this approach, an inducible expression system controlled by an external stimulus is desirable. Ideally such a system would not only mediate an “on/off” status for gene expression but would also permit limited expression of a gene at a defined level.

The methods and compositions of the present invention provide an inducible promoter that is regulated by the level of akt. The nucleic acid molecules, or fragments thereof, may also be utilized to control the expression of an expressible gene, e.g, a therapeutic gene, thereby regulating the amount of protein available to participate in a signaling pathway. Alterations in the physiological amount of the therapeutic gene may act to treat a disease associated with akt activity. In one embodiment, the nucleic acid molecules of the invention can be used to increase expression of a therapeutic gene. In another embodiment, the nucleic acid molecules of the invention may be used to decrease expression of a therapeutic gene in a population of target cells. It is to be understood that the expression of the inducible gene is regulated by an external levels of a protein that influences the PINK-1 promoter. For example, altered levels of akt may be used to “switch on” or “switch off” the PINK-1 promoter, therefore the transcription and expression of the expressible gene.

V. Production of Nucleic Acid Molecules

The nucleic acid molecules of the invention may be prepared by two general methods: (1) They may be synthesized from appropriate nucleotide triphosphates, or (2) they may be isolated from biological sources. Both methods utilize protocols well known in the art.

The availability of nucleotide sequence information, such as the full length cDNA having SEQ ID NO: 1, enables preparation of an isolated nucleic acid molecule of the invention by oligonucleotide synthesis. Synthetic oligonucleotides may be prepared by the phosphoramadite method employed in the Applied Biosystems 38A DNA Synthesizer or similar devices. The resultant construct may be purified according to methods known in the art, such as high performance liquid chromatography (HPLC). Long, double-stranded polynucleotides, such as a DNA molecule of the present invention, must be synthesized in stages, due to the size limitations inherent in current oligonucleotide synthetic methods. Thus, for example, a large double-stranded DNA molecule may be synthesized as several smaller segments of appropriate complementarity. Complementary segments thus produced may be annealed such that each segment possesses appropriate cohesive termini for attachment of an adjacent segment. Adjacent segments may be ligated by annealing cohesive termini in the presence of DNA ligase to construct the entire protein encoding sequence. A synthetic DNA molecule so constructed may then be cloned and amplified in an appropriate vector.

The nucleic acid molecules of the invention include cDNA, genomic DNA, RNA, and fragments thereof which may be single- or double-stranded. Thus, this invention provides oligonucleotides (sense or antisense strands of DNA or RNA, siRNA) having sequences capable of hybridizing with at least one sequence of a nucleic acid molecule of the present invention, such as selected segments of the cDNA having SEQ ID NO: 1.

The nucleic acids of the present invention may be maintained as DNA in any convenient cloning vector. In a preferred embodiment, clones are maintained in plasmid cloning/expression vector, such as pBluescript (Stratagene, La Jolla, Calif.), which is propagated in a suitable host cell.

VI. Production of Recombinant Proteins and Polypeptides

Recombinant proteins and polypeptides of the present invention may be prepared in a variety of ways, according to known methods. The protein may be purified from appropriate sources, e.g., human or animal cultured cells or tissues, by immunoaffinity purification. However, this is not a preferred method due to the small amounts of protein likely to be present in a given cell type at any time. The availability of nucleic acids molecules encoding the PINK-1 promoter enables production of the protein using in vitro expression methods known in the art. For example, a cDNA or gene may be cloned into an appropriate in vitro transcription vector, such a pSP64 or pSP65 for in vitro transcription, followed by cell-free translation in a suitable cell-free translation system, such as wheat germ or rabbit reticulocytes. In vitro transcription and translation systems are commercially available, e.g., from Promega Biotech, Madison, Wis. or BRL, Rockville, Md.

In one embodiment, the proteins or polypeptides encoded by the sequence shown in SEQ ID NO: 1 are produced by recombinant DNA techniques. For example, a nucleic acid molecule encoding the polypeptide is cloned into an expression vector, the expression vector introduced into a host cell and the polypeptide expressed in the host cell. The polypeptide can then be isolated from the cells by an appropriate purification scheme using standard protein purification techniques. Polypeptides can contain amino acids other than the 20 amino acids commonly referred to as the 20 naturally-occurring amino acids. Further, many amino acids, including the terminal amino acids, may be modified by natural processes, such as processing and other post-translational modifications, or by chemical modification techniques well known in the art. Common modifications that occur naturally in polypeptides are described in basic texts, detailed monographs, and the research literature, and they are well known to those of skill in the art.

Accordingly, the polypeptides also encompass derivatives or analogs in which a substituted amino acid residue is not one encoded by the genetic code, in which a substituent group is included, in which the mature polypeptide is fused with another compound, such as a compound to increase the half-life of the polypeptide (for example, polyethylene glycol), or in which the additional amino acids are fused to the mature polypeptide, such as a leader or secretory sequence or a sequence for purification of the mature polypeptide or a pro-protein sequence.

Known modifications include, but are not limited to, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphatidylinositol, cross-linking, cyclization, disulfide bond formation, demethylation, formation of covalent crosslinks, formation of cystine, formation of pyroglutamate, formylation, gamma carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination.

Such modifications are well-known to those of skill in the art and have been described in great detail in the scientific literature. Several particularly common modifications, glycosylation, lipid attachment, sulfation, gamma-carboxylation of glutamic acid residues, hydroxylation and ADP-ribosylation, for instance, are described in most basic texts, such as Proteins—Structure and Molecular Properties, 2nd Ed., T. E. Creighton, W. H. Freeman and Company, New York (1993). Many detailed reviews are available on this subject, such as by Wold, F., Posttranslational Covalent Modification of Proteins, B. C. Johnson, Ed., Academic Press, New York 1-12 (1983); Seifter et al., (1990) Meth. Enzymol. 182: 626-646) and Rattan et al., (1992) Ann. N.Y. Acad. Sci. 663:48-62.

As is also well known, polypeptides are not always entirely linear. For instance, polypeptides may be branched as a result of ubiquitination, and they may be circular, with or without branching, generally as a result of post-translation events, including natural processing event and events brought about by human manipulation which do not occur naturally. Circular, branched and branched circular polypeptides may be synthesized by non-translational natural processes and by synthetic methods.

Modifications can occur anywhere in a polypeptide, including the peptide backbone, the amino acid side-chains and the amino or carboxyl termini. Blockage of the amino or carboxyl group in a polypeptide, or both, by a covalent modification, is common in naturally-occurring and synthetic polypeptides. For instance, the amino terminal residue of polypeptides made in E. coli, prior to proteolytic processing, almost invariably will be N-formylmethionine.

The modifications can be a function of how the protein is made. For recombinant polypeptides, for example, the modifications will be determined by the host cell posttranslational modification capacity and the modification signals in the polypeptide amino acid sequence. Accordingly, when glycosylation is desired, a polypeptide should be expressed in a glycosylating host, generally a eukaryotic cell. Insect cells often carry out the same posttranslational glycosylations as mammalian cells and, for this reason, insect cell expression systems have been developed to efficiently express mammalian proteins having native patterns of glycosylation. Similar considerations apply to other modifications. The same type of modification may be present in the same or varying degree at several sites in a given polypeptide. Also, a given polypeptide may contain more than one type of modification.

VII. Vectors

The nucleic acid molecules and polypeptides of the invention can be delivered to a cell using viral vectors or by using non-viral vectors. In a preferred embodiment, the invention uses adeno-associated viral (AAV) vectors comprising the PINK-1 promoter operably linked to an expressible gene for gene delivery. AAV vectors can be constructed using known techniques to provide at least the operatively linked components of control elements including a transcriptional initiation region, a exogenous nucleic acid molecule, a transcriptional termination region and at least one post-transcriptional regulatory sequence. The control elements are selected to be functional in the targeted cell. The resulting construct which contains the operatively linked components is flanked at the 5′ and 3′ region with functional AAV ITR sequences.

The nucleotide sequences of AAV ITR regions are known. The ITR sequences for AAV-2 are described, for example by Kotin et al. (1994) Human Gene Therapy 5:793-801; Berns “Parvoviridae and their Replication” in Fundamental Virology, 2nd Edition, (B. N. Fields and D. M. Knipe, eds.) The skilled artisan will appreciate that AAV ITR's can be modified using standard molecular biology techniques. Accordingly, AAV ITRs used in the vectors of the invention need not have a wild-type nucleotide sequence, and may be altered, e.g., by the insertion, deletion or substitution of nucleotides. Additionally, AAV ITRs may be derived from any of several AAV serotypes, including but not limited to, AAV-1, AAV-2, AAV-3, AAV-4, AAV-5, AAVX7, and the like. Furthermore, 5′ and 3′ ITRs which flank a selected nucleotide sequence in an AAV expression vector need not necessarily be identical or derived from the same AAV serotype or isolate, so long as the ITR's function as intended, i.e., to allow for excision and replication of the bounded nucleotide sequence of interest when AAV rep gene products are present in the cell.

The skilled artisan can appreciate that regulatory sequences can often be provided from commonly used promoters derived from viruses such as, polyoma, Adenovirus 2, cytomegalovirus and Simian Virus 40. Use of viral regulatory elements to direct expression of the protein can allow for high level constitutive expression of the protein in a variety of host cells. Ubiquitously expressing promoters can also be used include, for example, the early cytomegalovirus promoter Boshart et al. (1985) Cell 41:521-530, herpesvirus thymidine kinase (HSV-TK) promoter (McKnight et al. (1984) Cell 37: 253-262), β-actin promoters (e.g., the human β-actin promoter as described by Ng et al. (1985) Mol. Cell Biol. 5: 2720-2732) and colony stimulating factor-1 (CSF-1) promoter (Ladner et al. (1987) EMBO J. 6: 2693-2698).

Alternatively, the regulatory sequences of the AAV vector can direct expression of the gene preferentially in a particular cell type, i.e., tissue-specific regulatory elements can be used. Non-limiting examples of tissue-specific promoters which can be used include, central nervous system (CNS) specific promoters such as, neuron-specific promoters (e.g., the neurofilament promoter; Byrne and Ruddle (1989) Proc. Natl. Acad. Sci. USA 86:5473-5477) and glial specific promoters (Morii et al. (1991) Biochem. Biophys Res. Commun. 175: 185-191). Preferably, the promoter is tissue specific and is essentially not active outside the central nervous system, or the activity of the promoter is higher in the central nervous system that in other systems. For example, a promoter specific for the spinal cord, brainstem, (medulla, pons, and midbrain), cerebellum, diencephalon (thalamus, hypothalamus), telencephalon (corpus stratium, cerebral cortex, or within the cortex, the occipital, temporal, parietal or frontal lobes), or combinations, thereof. The promoter may be specific for particular cell types, such as neurons or glial cells in the CNS. If it is active in glial cells, it may be specific for astrocytes, oligodentrocytes, ependymal cells, Schwann cells, or microglia. If it is active in neurons, it may be specific for particular types of neurons, e.g., motor neurons, sensory neurons, or interneurons. Preferably, the promoter is specific for cells in particular regions of the brain, for example, the cortex, stratium, nigra and hippocampus.

Suitable neuronal specific promoters include, but are not limited to, CMV/CBA, neuron specific enolase (NSE) (Olivia et al. (1991) Genomics 10: 157-165, GenBank Accession No: X51956), and human neurofilament light chain promoter (NEFL) (Rogaev et al. (1992) Hum. Mol. Genet. 1: 781, GenBank Accession No: L04147). Glial specific promoters include, but are not limited to, glial fibrillary acidic protein (GFAP) promoter (Morii et al. (1991) Biochem. Biophys Res. Commun. 175: 185-191, GenBank Accession No:M65210), S100 promoter (Morii et al. (1991) Biochem. Biophys Res. Commun. 175: 185-191, GenBank Accession No: M65210) and glutamine synthase promoter (Van den et al. (1991) Biochem. Biophys. Acta. 2: 249-251, GenBank Accession No: X59834). In a preferred embodiment, the gene is flanked upstream (i.e., 5′) by the neuron specific enolase (NSE) promoter. In another preferred embodiment, the gene of interest is flanked upstream (i.e., 5′) by the elongation factor 1 alpha (EF) promoter.

The AAV vector harboring the nucleotide sequence encoding a protein of interest, e.g., PINK-1, and a post-transcriptional regulatory sequence (PRE) flanked by AAV ITRs, can be constructed by directly inserting the nucleotide sequence encoding the protein of interest and the PRE into an AAV genome which has had the major AAV open reading frames (“ORFs”) excised therefrom. Other portions of the AAV genome can also be deleted, as long as a sufficient portion of the ITRs remain to allow for replication and packaging functions. These constructs can be designed using techniques well known in the art. (See, e.g., Lebkowski et al. (1988) Molec. Cell. Biol. 8:3988-3996; Vincent et al. (1990) Vaccines 90 (Cold Spring Harbor Laboratory Press); Carter (1992) Current Opinion in Biotechnology 3:533-539; Muzyczka (1992) Current Topics in Microbiol. and Immunol. 158:97-129; Kotin (1994) Human Gene Therapy 5:793-801; Shelling et al. (1994) Gene Therapy 1:165-169; and Zhou et al. (1994) J. Exp. Med. 179:1867-1875).

Alternatively, AAV ITRs can be excised from the viral genome or from an AAV vector containing the same and fused 5′ and 3′ of a selected nucleic acid construct that is present in another vector using standard ligation techniques, such as those described in Sambrook et al., Supra. Several AAV vectors are available from the American Type Culture Collection (“ATCC”) under Accession Numbers 53222, 53223, 53224, 53225 and 53226.

In order to produce recombinant AAV particles, an AAV vector can be introduced into a suitable host cell using known techniques, such as by transfection. A number of transfection techniques are generally known in the art. See, e.g., Graham et al. (1973) Virology, 52:456, Sambrook et al. (1989) Molecular Cloning, a laboratory manual, Cold Spring Harbor Laboratories, N.Y., Davis et al. (1986) Basic Methods in Molecular Biology, Elsevier, and Chu et al. (1981) Gene 13:197. Particularly suitable transfection methods include calcium phosphate co-precipitation (Graham et al. (1973) Virol. 52:456-467), direct micro-injection into cultured cells (Capecchi (1980) Cell 22:479-488), electroporation (Shigekawa et al. (1988) BioTechniques 6:742-751), liposome mediated gene transfer (Mannino et al. (1988) BioTechniques 6:682-690), lipid-mediated transduction (Felgner et al. (1987) Proc. Natl. Acad. Sci. USA 84:7413-7417), and nucleic acid delivery using high-velocity microprojectiles (Klein et al. (1987) Nature 327:70-73).

Suitable host cells for producing recombinant AAV particles include, but are not limited to, microorganisms, yeast cells, insect cells, and mammalian cells, that can be, or have been, used as recipients of a exogenous nucleic acid molecule. Thus, a “host cell” as used herein generally refers to a cell which has been transfected with an exogenous nucleic acid molecule. The host cell includes any eukaryotic cell or cell line so long as the cell or cell line is not incompatible with the protein to be expressed, the selection system chosen or the fermentation system employed. Non-limiting examples include CHO dhfr-cells (Urlaub and Chasin (1980) Proc. Natl. Acad. Sci. USA 77:4216-4220), 293 cells (Graham et al. (1977) J. Gen. Virol. 36: 59) or myeloma cells like SP2 or NSO (Galfre and Milstein (1981) Meth. Enzymol. 73(B):3-46).

In one embodiment, cells from the stable human cell line, 293 (readily available through, e.g., the ATCC under Accession No. ATCC CRL1573) are preferred in the practice of the present invention. Particularly, the human cell line 293, which is a human embryonic kidney cell line that has been transformed with adenovirus type-5 DNA fragments (Graham et al. (1977) J. Gen. Virol. 36:59), and expresses the adenoviral E1a and E1b genes (Aiello et al. (1979) Virology 94:460). The 293 cell line is readily transfected, and provides a particularly convenient platform in which to produce rAAV virions.

Host cells containing the above-described AAV vectors must be rendered capable of providing AAV helper functions in order to replicate and encapsidate the expression cassette flanked by the AAV ITRs to produce recombinant AAV particles. AAV helper functions are generally AAV-derived coding sequences which can be expressed to provide AAV gene products that, in turn, function in trans for productive AAV replication. AAV helper functions are used herein to complement necessary AAV functions that are missing from the AAV vectors. Thus, AAV helper functions include one, or both of the major AAV open reading frames (ORFs), namely the rep and cap coding regions, or functional homologues thereof.

The AAV rep coding region of the AAV genome encodes the replication proteins Rep 78, Rep 68, Rep 52 and Rep 40. These Rep expression products have been shown to possess many functions, including recognition, binding and nicking of the AAV origin of DNA replication, DNA helicase activity and modulation of transcription from AAV (or other exogenous) promoters. The Rep expression products are collectively required for replicating the AAV genome. The AAV cap coding region of the AAV genome encodes the capsid proteins VP1, VP2, and VP3, or functional homologues thereof. AAV helper functions can be introduced into the host cell by transfecting the host cell with an AAV helper construct either prior to, or concurrently with, the transfection of the AAV vector comprising the expression cassette, AAV helper constructs are thus used to provide at least transient expression of AAV rep and/or cap genes to complement missing AAV functions that are necessary for productive AAV infection. AAV helper constructs lack AAV ITRs and can neither replicate nor package themselves. These constructs can be in the form of a plasmid, phage, transposon, cosmid, virus, or virion. A number of AAV helper constructs have been described, such as the commonly used plasmids pAAV/Ad and pIM29+45 which encode both Rep and Cap expression products. (See, e.g., Samulski et al. (1989) J. Virol. 63:3822-3828; and McCarty et al. (1991) J. Virol. 65:2936-2945). A number of other vectors have been described which encode Rep and/or Cap expression products. See, e.g., U.S. Pat. No. 5,139,941.

As a consequence of the infection of the host cell with a helper virus, the AAV Rep and/or Cap proteins are produced. The Rep proteins also serve to duplicate the AAV genome. The expressed Cap proteins assemble into capsids, and the AAV genome is packaged into the capsids. This results the AAV being packaged into recombinant AAV particles comprising the expression cassette. Following recombinant AAV replication, recombinant AAV particles can be purified from the host cell using a variety of conventional purification methods, such as CsCl gradients. The resulting recombinant AAV particles are then ready for use for gene delivery to various cell types.

Alternatively, a vector of the invention can be a virus other than the adeno-associated virus, or portion thereof, which allows for expression of a nucleic acid molecule introduced into the viral nucleic acid. For example, replication defective retroviruses, adenoviruses and lentivirus can be used. Protocols for producing recombinant retroviruses and for infecting cells in vitro or in vivo with such viruses can be found in Current Protocols in Molecular Biology, Ausubel et al. (eds.) Greene Publishing Associates, (1989), Sections 9.10-9.14 and other standard laboratory manuals. Examples of suitable retroviruses include pLJ, pZIP, pWE and pEM which are well known to those skilled in the art. Examples of suitable packaging virus lines include Crip, Cre, 2 and Am. The genome of adenovirus can be manipulated such that it encodes and expresses the protein of interest but is inactivated in terms of its ability to replicate in a normal lytic viral life cycle. See e.g., Berkner et al. (1988) BioTechniques 6:616; Rosenfeld et al. (1991) Science 252:431-434; and Rosenfeld et al. (1992) Cell 68:143-155. Suitable adenoviral vectors derived from the adenovirus strain Ad type 5 dl324 or other strains of adenovirus (e.g., Ad2, Ad3, Ad7 etc.) are well known to those skilled in the art.

Alternatively, the vector can be delivered using a non-viral delivery system. This includes delivery of the vector to the desired tissues in colloidal dispersion systems that include, for example, macromolecule complexes, nanocapsules, microspheres, beads, and lipid-based systems including oil-in-water emulsions, micelles, mixed micelles, and liposomes.

Liposomes are artificial membrane vesicles which are useful as delivery vehicles in vitro and in vivo. In order for a liposome to be an efficient gene transfer vehicle, the following characteristics should be present: (1) encapsulation of the genetic material at high efficiency while not compromising the biological activity; (2) preferential and substantial binding to a target cell in comparison to non-target cells; (3) delivery of the aqueous contents of the vesicle to the target cell cytoplasm at high efficiency; and (4) accurate and effective expression of genetic information (Mannino, et al. (1988) Biotechniques, 6:682). Examples of suitable lipids liposomes production include phosphatidyl compounds, such as phosphatidylglycerol, phosphatidylcholine, phosphatidylserine, phosphatidylethanolamine, sphingolipids, cerebrosides, and gangliosides. Additional examples of lipids include, but are not limited to, polylysine, protamine, sulfate and 3b -[N—(N′,N′dimethylaminoethane)carbamoyl]cholesterol.

Alternatively, the vector can be coupled with a carrier for delivery Exemplary and preferred carriers are keyhole limpet hemocyanin (KLH) and human serum albumin. Other carriers may include a variety of lymphokines and adjuvants such as INF, IL-2, IL-4, IL-8 and others. Means for conjugating a peptide to a carrier protein are well known in the art and include glutaraldehyde, m-maleimidobenzoyl- N-hydroxysuccinimide ester, carbodiimyde and bis-biazotized benzidine. The vector can be conjugated to a carrier by genetic engineering techniques that are well known in the art. (See e.g., U.S. Pat. Nos. 4,608,251; 4,601,903; 4,599,231; 4,599,230; 4,596,792; and 4,578,770).

In one embodiment, particle-mediated delivery using a gene-gun can be used as a method to deliver the vector. Suitable particles for gene gun-based delivery of include gold particles. In one embodiment, the vector can be delivered as naked DNA. Gene gun based delivery is described, for example by, Braun et al. (1999) Virology 265:46-56; Drew et al. (1999) Vaccine 18:692-702;.Degano et al. (1999) Vaccine 18:623-632; and Robinson (1999) Int J Mol Med 4:549-555; Lai et al. (1998) Crit Rev Immunol 18:449-84; See e.g., Accede et al. (1991) Nature 332: 815-818; and Wolff et al. (1990) Science 247:1465-1468 Murashatsu et al., (1998) Int. J. Mol. Med. 1: 55-62; Agracetus et al. (1996) J. Biotechnol. 26: 37-42; Johnson et al. (1993) Genet. Eng. 15: 225-236). Also within the scope of the invention is the delivery of the vector in one or more combinations of the above delivery methods.

VIII. Delivery Systems

Delivery systems include methods of in vitro, in vivo and ex vivo delivery of the vector. For in vivo delivery, the vector can be administered to a subject in a pharmaceutically acceptable carrier. The term “pharmaceutically acceptable carrier”, as used herein, refers to any physiologically acceptable carrier for in vivo administration of the vectors of the present invention. Such carriers do not induce an immune response harmful to the individual receiving the composition, and are discussed below.

In one embodiment, vector can be distributed throughout a wide region of the CNS, by injecting the vector into the cerebrospinal fluid, e.g., by lumbar puncture (See e.g., Kapadia et al. (1996) Neurosurg 10: 585-587).

Alternatively, precise delivery of the vector into specific sites of the brain, can be conducted using stereotactic microinjection techniques. For example, the subject being treated can be placed within a stereotactic frame base (MRI-compatible) and then imaged using high resolution MRI to determine the three-dimensional positioning of the particular region to be treated. The MRI images can then be transferred to a computer having the appropriate stereotactic software, and a number of images are used to determine a target site and trajectory for antibody microinjection. The software translates the trajectory into three-dimensional coordinates that are precisely registered for the stereotactic frame. In the case of intracranial delivery, the skull will be exposed, burr holes will be drilled above the entry site, and the stereotactic apparatus used to position the needle and ensure implantation at a predetermined depth. The vector can be delivered to regions, such as the cells of the spinal cord, brainstem, (medulla, pons, and midbrain), cerebellum, diencephalon (thalamus, hypothalamus), telencephalon (corpus stratium, cerebral cortex, or within the cortex, the occipital, temporal, parietal or frontal lobes), or combinations, thereof. In another preferred embodiment, the vector is delivered using other delivery methods suitable for localized delivery, such as localized permeation of the blood-brain barrier. Particularly preferred delivery methods are those that deliver the vector to regions of the brain that require modification.

IX. Screening and Diagnostic Assays

The nucleic acids of the invention (including variants, fragments and analogs thereof) are useful for screening assays, in both in vitro and in vivo cell-based systems. Cell-based systems that involve recombinant host cells expressing the an expressible gene (e.g., a marker gene, e.g., luciferase) under the control of the PINK-1 promoter. The effect of the drugs that alter the promoter activity can be investigated by examining the level of luciferase expression.

The PINK-1 promoter operably linked to an expressible gene can be used to identify compounds that modulate PINK-1 promoter activity. This can be used in high-throughput screens to assay candidate compounds. Compounds can be identified that activate or inactivate the PINK-1 promoter activity to a desired degree. Any of the biological or biochemical functions mediated by the akt receptor can be used as an endpoint assays. These include all of the biochemical or biochemical/biological events described herein, in the references cited herein, incorporated by reference for these endpoint assay targets, and other functions known to those of ordinary skill in the art.

In vitro techniques for detection of the expressible gene include enzyme linked immunosorbent assays (ELISAs), Western blots, immunoprecipitations and immunofluorescence. Alternatively, the protein can be detected in vivo in a subject by introducing into the subject a labeled anti-receptor antibody. For example, the antibody can be labeled with a radioactive marker whose presence and location in a subject can be detected by standard imaging techniques.

The methods and compositions of the invention may also be used for diagnostic purposes using a marker gene (e.g., luciferase) operably linked to the PINK-1 promoter. Alterations in the akt level would switch on or switch off the activity of the PINK-1 promoter, which in turn would switch on or switch off the expression of the luciferase marker gene. The luciferase marker gene can be detected in cells using CAT scans or MRI scans.

The methods and compositions of the invention may also be used with multiple vectors with different expressible genes. For example, a first vector with a first PINK-1 promoter operably linked to a reporter gene, e.g., luciferase, and a second vector with a second PINK-1 promoter operably linked to a therapeutic gene, such that an alteration in normal cellular akt levels switches on or off the reporter gene for diagnostic purposes, and switches on or off the therapeutic gene, e.g., a growth factor (e.g., gdnf) to ameliorate the disorder resulting from the altered akt level.

X. Pharmaceutical Compositions and Pharmaceutical Administration

The vector of the invention can be incorporated into pharmaceutical compositions suitable for administration to a subject. Typically, the pharmaceutical composition comprises the vector or sequence of the invention and a pharmaceutically acceptable carrier. As used herein, “pharmaceutically acceptable carrier” includes any and all solvents, dispersion media, coatings, antibacterial and antifungal agents, isotonic and absorption delaying agents, and the like that are physiologically compatible. Examples of pharmaceutically acceptable carriers include one or more of water, saline, phosphate buffered saline, dextrose, glycerol, ethanol and the like, as well as combinations thereof. In many cases, it will be preferable to include isotonic agents, for example, sugars, polyalcohols such as mannitol, sorbitol, or sodium chloride in the composition. Pharmaceutically acceptable carriers may further comprise minor amounts of auxiliary substances such as wetting or emulsifying agents, preservatives or buffers, which enhance the shelf life or effectiveness of the composition.

The compositions of this invention may be in a variety of forms. These include, for example, liquid, semi-solid and solid dosage forms, such as liquid solutions (e.g., injectable and infusible solutions), dispersions or suspensions, tablets, pills, powders, liposomes and suppositories. The preferred form depends on the intended mode of administration and therapeutic application. Typical preferred compositions are in the form of injectable or infusible solutions, such as compositions similar to those used for passive immunization of humans. The preferred mode of administration is parenteral (e.g., intravenous, subcutaneous, intraperitoneal, intramuscular). In one embodiment, the vector is administered by intravenous infusion or injection. In another embodiment, the vector is administered by intramuscular or subcutaneous injection. In another embodiment, the vector is administered perorally. In the most preferred embodiment, the vector is delivered to a specific location using stereostatic delivery.

Therapeutic compositions typically must be sterile and stable under the conditions of manufacture and storage. The composition can be formulated as a solution, microemulsion, dispersion, liposome, or other ordered structure suitable to high drug concentration. Sterile injectable solutions can be prepared by incorporating the active compound (i.e., antigen, antibody or antibody portion) in the required amount in an appropriate solvent with one or a combination of ingredients enumerated above, as required, followed by filtered sterilization.

Generally, dispersions are prepared by incorporating the active compound into a sterile vehicle that contains a basic dispersion medium and the required other ingredients from those enumerated above. In the case of sterile, lyophilized powders for the preparation of sterile injectable solutions, the preferred methods of preparation are vacuum drying and spray-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

The vector of the present invention can be administered by a variety of methods known in the art. As will be appreciated by the skilled artisan, the route and/or mode of administration will vary depending upon the desired results. In certain embodiments, the active compound may be prepared with a carrier that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978. The pharmaceutical compositions of the invention may include a “therapeutically effective amount” or a “prophylactically effective amount” of the vectors of the invention. A “therapeutically effective amount” refers to an amount effective, at dosages and for periods of time necessary, to achieve the desired therapeutic result. A therapeutically effective amount of the vector may vary according to factors such as the disease state, age, sex, and weight of the individual, and the ability of the vector to elicit a desired response in the individual. A therapeutically effective amount is also one in which any toxic or detrimental effects of the vector are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

Dosage regimens may be adjusted to provide the optimum desired response (e.g., a therapeutic or prophylactic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic or prophylactic effect to be achieved, and (b) the limitations inherent in the art of compounding such an active compound for the treatment of sensitivity in individuals.

One skilled in the art will appreciate further features and advantages of the invention based on the above-described embodiments. Accordingly, the invention is not to be limited by what has been particularly shown and described, except as indicated by the appended claims. All publications and references cited herein are expressly incorporated herein by reference in their entirety.

### EXAMPLES

(A) Examples Involving the PINK-1 Promoter

### Example 1

### Methods and Materials

(i) Construction of PTEN-Overexpressing Cell Lines

A full length human PTEN cDNA (a gift from A. Yung, M.D. Anderson Cancer Center, and shown in FIG. 2) was cloned and transfected into PC12 and U87 cells using FuGene 6 (Roche). Four weeks following hygromycin selection (250 μg/ml) individual clones were expanded and analyzed for PTEN expression. Control lines were also used for the experiments in this study.

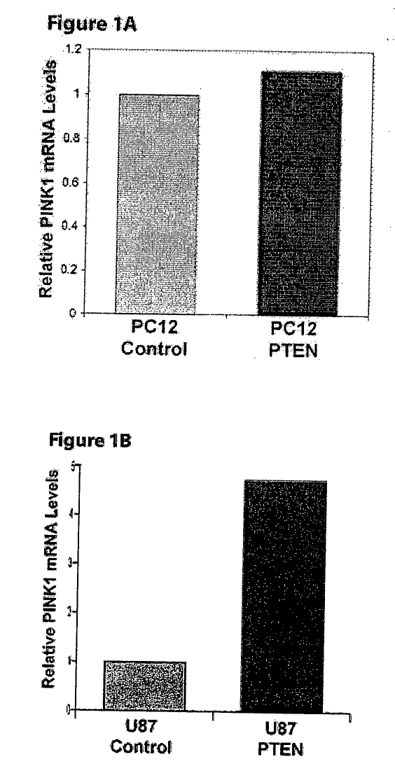

The constructs were used to transfect cell lines. FIG. 1 shows PINK1 levels in stable cell lines overexpressing PTEN. Previously, we reported on the effect of PTEN on gene expression in these cells (Musatov et al. Proc Natl Acad Sci USA. 2004 101(10):3627-31). Using qPCR, there was no difference in PTEN levels in the PC12 isolates, while U87 human glioma cells overexpressing PTEN had roughly 5-fold higher levels of PINK1 mRNA. The lack of effect on PC12 cells may simply represent a difference in regulation between human and rodent cells, but this may also reflect more complex regulation of PINK1 depending upon cell-type.

(ii) Cloning PINK1 Promoter

Using RT-PCR the PINK1 promoter was cloned from human genomic cDNA. The nucleotide sequence of the PINK-1 promoter is shown in FIG. 2 and designated SEQ ID NO: 1. The various domains of the PINK-1 promoter are indicated in capital letters in the following sequence: NF-kB, CRE-BP, Interferon Response Stimulated Element, Interferon Regulatory Factor 2, and NF-kB, followed by the known PINK-1 transcript.

(iii) Western Blotting

Blots were stained with various antibodies for PTEN, AKT, and (Cell Signaling).

(iv) Quantitative PCR (Q-PCR)

Real-time PCR was performed using SYBR Green Master Mix (Applied Biosystems) on an ABI Prism 7000 Sequence Detection System (Applied Biosystems).

(v) Promoter Analysis

HEK293 cells were transfected with PINK1pr2220 and various controls for 48 hrs. Promoter activity was determined using Dual Glo Luciferase Assay system (Promega) and normalized against renilla luciferase.

### Example 2

### Characterization of the PINK-1 Promoter

To characterize the effects of the PINK-1 promoter, a number of experiments were conducted and the level of promoter expression, activity and effect were analyzed.

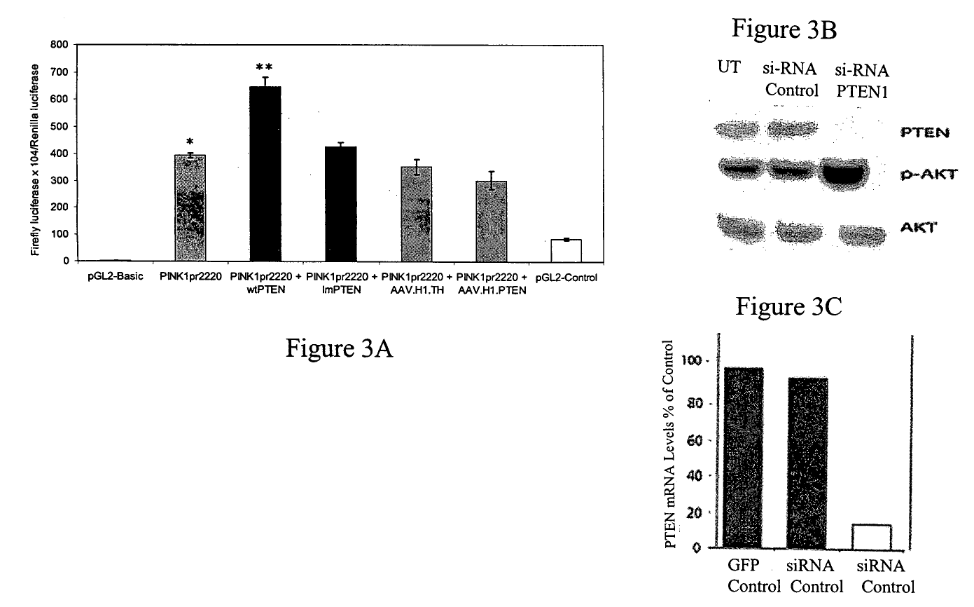

The effects of PTEN on the isolated PINK1 promoter fragment were analyzed and summarized in FIG. 3. In FIG. 3(A) the results are represented as the level of firefly luciferase activity driven by the PINK1 promoter normalized against renilla luciferase activity from a second plasmid used to control for transfection variability. Each experiment was replicated in triplicate, and the entire study was replicated on at least two separate occasions. Transfection of the promoter alone revealed significant promoter activity compared to cells transfected with a construct with either no promoter (pGL2-basic) or an SV40 promoter (pGL2-control) (*p<0.05; two-tailed t-test). Co-transfection with PTEN induced PINK1 promoter activity from 65% to 5 fold in various replicate experiments compared with PINK1 alone (**p<0.05; two-tailed t-test), while a PTEN point-mutant which lacks lipid phosphatase activity (1 mPTEN) had no effect. Inhibition of PTEN expression using a construct expressing an anti-PTEN RNAi molecule (FIG. 3B) caused a slight increase in activated, phosphorylated AKT on Western blot (FIG. 3C) but this did not alter PINK1 promoter activity (FIG. 3A, AAVH1PTEN).

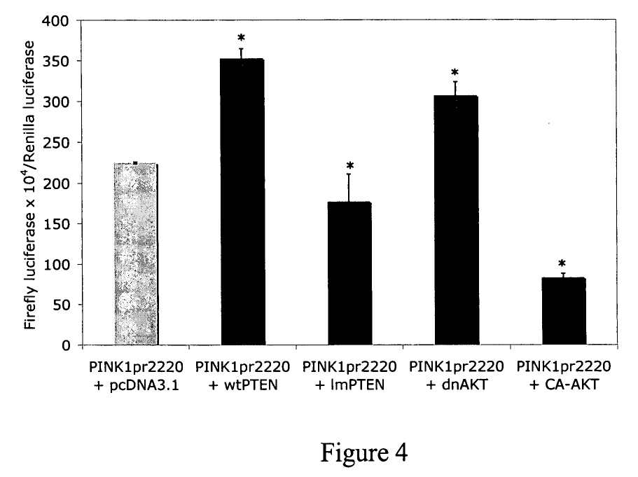

The effect of AKT activity was investigated and the results shown in FIG. 4. FIG. 4 shows that alterations in AKT Activity dynamically regulate the human PINK1 promoter. Lack of PINK1 promoter induction by PTEN lacking lipid phosphatase activity suggests an AKT-mediated mechanism. Co-transfection of a dominant-negative AKT (dnAKT) mutant induced PINK1 promoter activity to levels comparable to PTEN. Although PTEN blockade did not inhibit PINK1 promoter activity, co-transfection with a consitutively-active AKT (cAKT) did reduce PINK1 promoter activity roughly 2-fold. Data is the result of triplicate experiments analyzed relative to promoter activity of PINK1pr2220 alone (*p<0.05; two-tailed t-test).

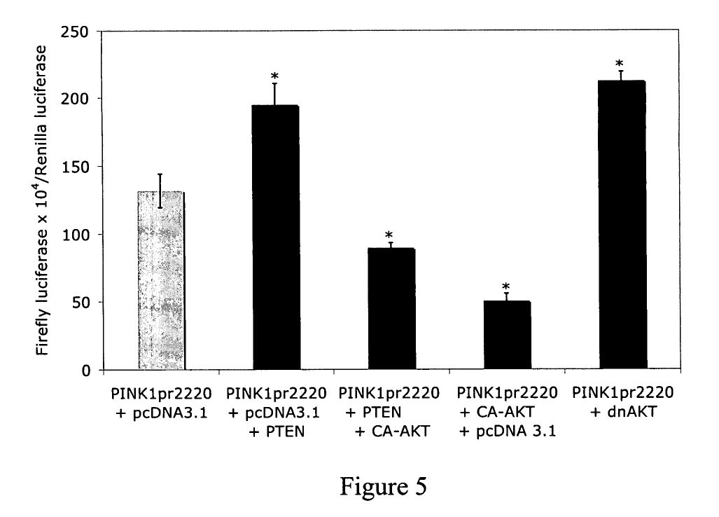

FIG. 5 shows that constitutively active AKT overcome the effect of PTEN on the PINK1 promoter. Co-transfection of both cAKT and PTEN resulted in PINK1 promoter activity which was below baseline near levels resulting from cAKT transfection alone. The slight increase may reflect the effect of PTEN on endogenous cellular AKT. All plasmids were kept at constant amounts and ratios, with empty plasmid replacing constructs not used for individual studies. Data is the result of triplicate experiments analyzed relative to promoter activity of PINK1pr2220 alone (*p<0.05; two-tailed t-test).

Collectively, these results demonstrate that a 2220bp sequence upstream of the reported PINK1 transcriptional start site has significant promoter activity. In addition, PTEN induces activity of this promoter by up to 5-fold, consistent with effects on endogenous cellular PINK1 expression in ovarian cancer and human glioma cells. This supports the conclusion that this sequence does contain the PINK1 promoter.

The mechanism of PTEN action on PINK1 promoter activity appears to be mediated by inhibition of AKT. Loss of this activity fails to induce PINK1 while dominant negative AKT is equally effective as PTEN. Although inhibition of PTEN failed to alter PINK1 promoter activity, constitutively active PTEN significantly inhibits PINK1 promoter activity. Since PTEN inhibition causes only a mild activation of AKT, this suggests that significant alterations in AKT activity can dynamically regulate PINK1 promoter activity. The regulation of PINK1 promoter activity by AKT suggests that physiological stimuli which influence AKT levels and/or activity may also regulate PINK1 expression.

(B) Examples Involving PTEN

### Example 3

### Methods and Materials

(i) Cell Culture and Treatments.

SH-SY5Y cells were maintained in Dulbecco's modified medium (DMEM) supplemented with 10% FBS, 100 IU/mL penicillin and 100 mg/mL streptomycin at 37 oC under 5% CO2. Toxins (Sigma) were freshly prepared and added to the cultures and incubated for various lengths of time.

(ii) Western Blotting.

Blots were incubated with antibodies against PTEN, Phospho-PTEN, and Caspase-3 (Cell Signaling) followed by secondary antibodies and enhanced chemiluminescent detection (Amersham Biosciences).

(iii) SiRNA Experiments.

PTEN SMARTpool siRNA reagent was purchased from Upstate. Cells were transfected with Lipofectamine Reagent (Invitrogen) according to the manufacturer's protocol in the presence of siRNA. Non-specific control SMARTpool siRNA (Upstate) was included as a control.

(iv) 6-OHDA Microinjections.

Rats were anesthetized with ketamine and xylazine solution (5:1.1) and positioned in the stereotaxic frame. Lesions were produced by unilaterally microinjecting 2 μL of 6-OHDA (1 μg/μL) with 0.02% ascorbic acid (Sigma) dissolved in 0.9% saline into the medial forebrain bundle (MFB) with a Hamilton syringe with an injection rate of 2 μL/min (n=5). Saline (2 μL) was injected on the opposite side as the matched control. Rats were sacrificed 24 hours after injection.

(v) Cell Survival and Apoptosis Assay.

To measure cell viability and apoptosis CellTiter 96 Aqueous One Solution Cell Proliferation Assay (Promega) and propidium iodide (Sigma) were used, respectively.

### Example 4

### Alterations in the PTEN Tumor Suppressor Mediate Neurotoxicity in Dopaminergic Cells

Parkinson s Disease (PD) is characterized by dopaminergic neuronal degeneration in the substantia nigra pars compacta (SNc). PTEN is a potent tumor suppressor, which is also expressed in most normal neurons and has a variety of known functions, including inhibition of the PI3Kinase/AKT cell survival pathway. Therefore, we hypothesized that while PTEN might prevent tumor formation or progression, these same properties could also make aging neurons more susceptible to degenerative processes such as PD. We now report that PTEN mediates at least in part the effects of the neurotoxin 6-hydroxydopamine (6-OHDA), which specifically causes the death of dopaminergic neurons in vivo and in vitro. Rats lesioned with 6-OHDA in the medial forebrain bundle have decreased levels of phosphorylated PTEN (P-PTEN) in the SNc when compared with saline controls. Furthermore, human neuroblastoma SH-SY5Y cells challenged with 6-OHDA showed a similar reduction in P-PTEN by both western blot and immunoprecipitation.

Since phosphorylation inhibits PTEN activity, this suggests that the 6-OHDA insult increased PTEN activity. These changes correlated directly with both the increase in caspase activation at 6 hrs and eventual cell death at 24 hrs. Inhibition of endogenous PTEN using RNA interference (RNAi) resulted in increased cell survival and decreased apoptosis at every dose of 6-OHDA compared with matched controls. For in vivo manipulation, we have now generated an adeno-associated Virus vector containing the PTEN RNAi construct, which appears to reduce PTEN mRNA levels by almost 90%.

These data suggest that alterations in activity of the PTEN tumor suppressor may mediate some of the neurotoxic effects of 6-OHDA, and strategies to block PTEN expression or function in dopaminergic neurons may provide novel gene therapy for Parkinson s disease.

The experiments and results are as follows:

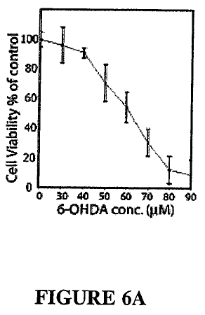

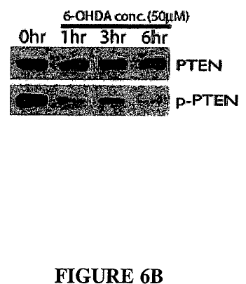

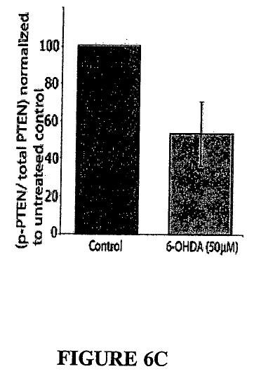

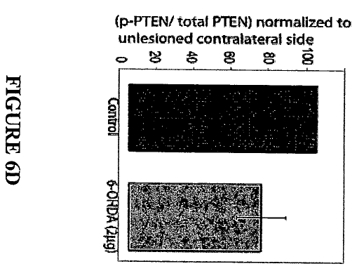

6-OHDA induced cell death and modulation of PTEN was investigated and the results shown in FIGS. 6A-D. FIG. 6A shows 6-OHDA reduced cell viability of human neuroblastoma SH-SY5Y in a dose dependent manner. FIG. 6B shows the results of cells were treated at various time points with 6-OHDA. PTEN and phosphorylated PTEN (p-PTEN) were determined by using western blot analysis. FIG. 6C show PTEN activity was determined by comparing the ratio of p-PTEN/total PTEN. A decrease in p-PTEN/total PTEN is correlated with higher PTEN activity. Results were analyzed by Image J software and p-PTEN/total PTEN results were shown graphically. Human neuroblastoma cells treated with 6-OHDA (50 mM) for 6 hrs showed a lower p-PTEN/total PTEN ratio as determined by western blot (n=4). FIG. 6D shows that adult rats (n=4) were lesioned unilaterally by microinjecting 6-OHDA (2 μg) into the medial forebrain bundle (MFB). Substantia nigra pars compacta were dissected and harvested for western blot analysis as previously described. 6-OHDA treated side showed similar reductions in p-PTEN/total PTEN levels when compared to unlesioned contralateral side.

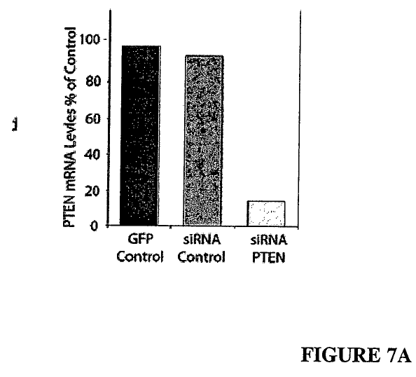

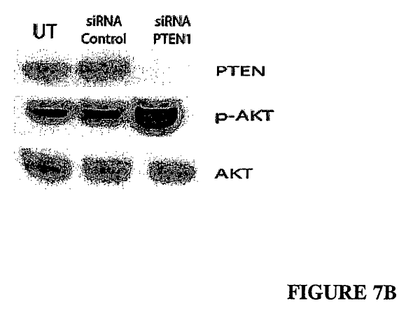

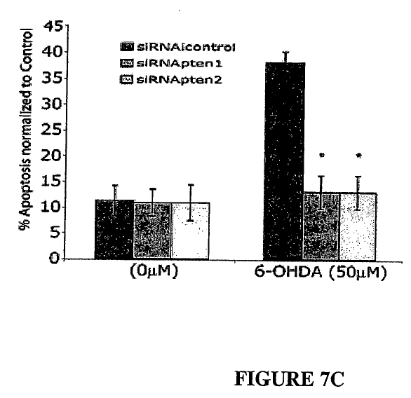

To investigate the effect of PTEN siRNA, PTEN siRNA plasmids reduced PTEN protein expression and mRNA levels in SH-SKN cells and reduced 6-OHDA induced apoptosis in vitro, as shown in FIGS. 7A-C. FIG. 7A shows adenoviral associated viral vector containing a construct for PTEN siRNA drastically reduced PTEN protein levels in SH-SKN cells when analyzed by western blot. Knocking down PTEN showed the physiologic effect of increasing the phosphorylated form of AKT, which has been correlated with greater AKT activity. FIG. 7B shows that this same construct reduced PTEN mRNA levels by 90% when analyzed by quantitative PCR. FIG. 7C shows SH-SY5Y cells transfected with two separate PTEN siRNA plasmids showed a significant reduction in apoptosis compared to the scrambled siRNA control as analyzed by propidium iodide staining. (*p<0.05; two-tailed t-test).

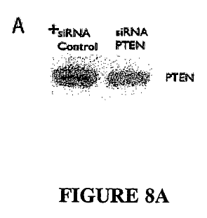

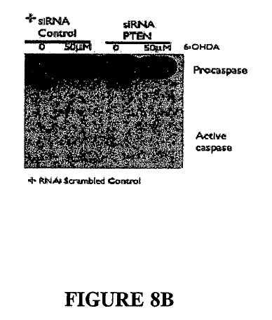

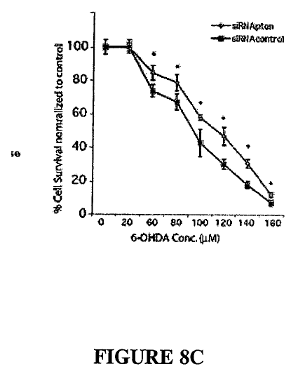

FIGS. 8A-C show that PTEN RNAi oligos modestly reduced PTEN protein expression in SH-SY5Y cells and increased viability after 6-OHDA insult. In FIG. 8A PTEN SMARTpool siRNA Reagent (Upstate) was transfected in SHSY5Y cells and reduced PTEN protein levels by 50% when analyzed by western blot, ash shown in FIG. 8B. After challenging SH-SY5Y cells with 6-OHDA for 6hrs, PTEN RNAi transfected cells reduced the cleavage of the Caspase-3 active metabolite. FIG. 8C shows that SH-SY5Y cells transfected with PTEN siRNA oligos resulted in an increase in cell viability at every dose of 6-OHDA compared to the matched control as analyzed by Cell Titer proliferation assay (Promega). (*p<0.05; two-tailed t-test).

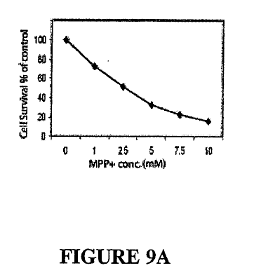

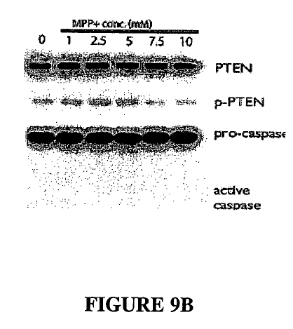

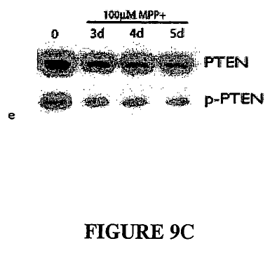

FIGS. 9A-C shows that high dose MPP+induced SH-SY5Y cell death yet PTEN was not modulated at this dose, however regulation of PTEN occurred at a low dose bolus. FIG. 9A shows that 24 hr treatment of MPP+ reduced cell viability of human neuroblastoma SH-SY5Y in a dose dependent manner. B) SH-SY5Y cells were treated for 24 hrs with escalating doses of MPP+ (mM). PTEN and phosphorylated PTEN (p-PTEN) levels were determined by using western blot analysis. PTEN activity was determined by comparing the ratio of p-PTEN/total PTEN. No change in p-PTEN/total PTEN was observed at 24 hrs. Caspase-3 active metabolites were not cleaved at these high doses, which is consistent with previous reports that showed high (mM) doses of MPP+ caused necrotic cell death. FIG. 9C shows human neuroblastoma cells treated with low dose MPP+ (100 μM), which has been shown to induce apoptosis, 1 for various time points and showed a reduction p-PTEN/total PTEN ratio as determined by western blot. This suggests PTEN is modulated only at apoptotic or low doses of MPP+.

Collectively, these results show that (P-PTEN/Total PTEN) levels decrease in the presence 6-OHDA treatment in vitro and in vivo. Also that knocking down PTEN with siRNA protects cells from 6-OHDA insult and reduces apoptotic cell death in vitro. The adeno-associated virus construct containing a PTEN RNAi, may be a valuable tool for in vivo studies. In addition, low dose MPP+, which has previously been shown to induce apoptosis, modulates PTEN activity.

What is claimed is:

1. A promoter sequence comprising SEQ ID NO: 1.

2. The promoter sequence of claim 1, wherein the promoter sequence is an inducible promoter sequence.

3. The promoter sequence of claim 2, wherein the inducible promoter sequence is responsive to altered Akt levels.

4. A promoter-driven protein expression system comprising a PINK-1 promoter sequence operably linked to an expressible gene.

5. The promoter system of claim 4, wherein the PINK-1 promoter sequence comprises a domain selected from the group consisting of a first NF-kB domain, CRE-BP domain, Interferon Response Stimulated Element, Interferon Regulatory Factor 2, and a second NF-kB domain.

6. The promoter system of claim 5, wherein the expressible gene is selected from the group consisting of a therapeutic gene and a reporter gene.

7. A vector comprising a nucleic acid comprising a PINK-1 promoter sequence operably linked to an expressible gene.

8. The vector of claim 7, wherein the expressible gene is selected from the group consisting of a therapeutic gene and a reporter gene.

9. A host cell transformed by the vector of claim 7.

10. A method of producing a recombinant protein comprising:
transforming a host cell with the vector of claim 7; and
expressing the expressible gene of said vector.

11. A method for ameliorating a disorder associated with a PI-3 kinase/Akt pathway in a subject comprising:
delivering a vector comprising a PINK-1 promoter operably linked to a therapeutic gene to the target site in the subject; and
expressing the therapeutic gene in the target site to ameliorate the disorder.

12. The method of claim 11, wherein the disorder associated with a PI-3 kinase/Akt pathway is selected form the group consisting of cardiovascular disorders, neurodegenerative disorders, cell proliferative disorders, cancers, and endocrine disorders.

13. A polypeptide comprising an amino acid sequence having a 60% or more homology with the amino acid sequence of SEQ. ID NO: 1, and which is responsive to altered Akt levels.

14. The polypeptide of claim 13, which is an inducible promoter.

15. The polypeptide of claim 14 having a 70% or more homology with the amino acid sequence of SEQ ID NO: 1.

16. The polypeptide of claim 14 having an 85% or more homology with the amino acid sequence of SEQ ID NO: 1.

17. The polypeptide of claim 14 having a 90% or more homology with the amino acid sequence of SEQ ID NO: 1.

18. The polypeptide of claim 14 having a 95% or more homology with the amino acid sequence of SEQ ID NO: 1.

19. The polypeptide of claim 14 having a 98% or more homology with the amino acid sequence of SEQ ID NO: 1.

20. A nucleic acid sequence coding for the polypeptide of claim 13.

21. A nucleic acid sequence coding for the polypeptide of claim 1.
